(19) United States
(12) Patent Application Publication
(10) Pub. No.: US 2023/0181923 A1
(43) Pub. Date: Jun. 15, 2023

(54) APPARATUS AND METHOD FOR TREATING SKIN LESIONS

(71) Applicant: Zerigo Health, Inc., San Diego, CA (US)

(72) Inventor: Frank D'Amelio, San Clemente, CA (US)

(21) Appl. No.: 17/546,650

(22) Filed: Dec. 9, 2021

(51) Int. Cl. A61N 5/06

(52) U.S. Cl. CPC A61N 5/0616; A61N 2005/0652; A61N 2005/0626; A61N 2005/0643

(57) ABSTRACT

An apparatus and method for treating a skin lesion on a user includes obtaining images of the user from a camera, determining an area of the user's body for treating the skin lesion, positioning an LED assembly comprising an LED array, and activating one or more LEDs in the LED array to treat the skin lesion.

### CROSS-REFERENCE TO OTHER APPLICATION

This application is being filed on the same day as the application identified by Attorney Docket Number 27471.0004, also assigned to the current applicant, which application is herein incorporated by reference in its entirety.

### BACKGROUND

One set of ailments afflicting people are skin lesions. One method for treating skin lesions involves applying light transmitted from light-emitting diodes (LEDs) to the skin lesions. Traditional methods of treating skin lesions with LEDs can result in the healthy, i.e., non-lesioned or improved, skin receiving unneeded/unwanted light (or, more broadly, radiation) transmitted from the LEDs. Applying LED radiation to healthy skin is undesirable for numerous reasons. For example, applying LED radiation to healthy skin results in discoloration of the healthy skin compared to the rest of the patient's skin, which patients generally dislike because they do not find it aesthetically pleasing. In addition, applying LED radiation to healthy skin results in an increased risk of unwanted skin conditions to the healthy skin, such as sunburn and skin cancer.

Traditional methods and systems for LED-based treatment of skin lesions are particularly problematic in patients with large skin lesions. One method used on patients with large skin lesions uses large LED radiation sources, which results in large portions of the patient's healthy skin receiving unwanted LED radiation. Alternative methods use smaller LED radiation sources. However, using smaller LED radiation sources, which minimize the amount of healthy skin exposed to LED radiation, requires performing numerous lengthy treatments to cover the entire skin lesion.

### BRIEF DESCRIPTION OF THE DRAWINGS

FIG. 1A is a diagram showing an LED-based skin treatment apparatus with bar-type LED assemblies, according to an embodiment of the present invention;

FIG. 1B is a diagram showing the LED-based skin treatment apparatus of FIG. 1A with a user in a seated position, according to an embodiment of the present invention;

FIG. 2 is a diagram showing an LED-based skin treatment apparatus with disc-type LED assemblies, according to an embodiment of the present invention;

FIG. 3 is a diagram showing a disc-type LED assembly, according to an embodiment of the present invention;

FIG. 4 is a flowchart showing a process for preparing to treat skin lesions, according to an embodiment of the present invention;

FIG. 5 is a flowchart showing a process for treating skin lesions, according to an embodiment of the present invention;

FIG. 6 is a diagram showing the LED-based skin treatment apparatus with bar-type LED assemblies during skin treatment, according to an embodiment of the present invention; and

FIGS. 7A and 7B are diagrams showing an LED-based skin treatment apparatus mounted on a wheeled baseplate in upright and folded positions, according to embodiments of the present invention.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

In addition to the problems noted above, some skin lesions may be located in hard-to-reach and hard-to-see locations. These locations may include the back, the buttocks, and the back of the upper thigh. The inventor has developed an apparatus and method for treating a skin lesion on a user that addresses these issues. The method includes obtaining images of the user from a camera, determining an area of the user's body for treating the skin lesion or lesions, positioning an LED assembly that includes an LED array, and activating one or more LEDs in the LED array to treat the skin lesion or lesions.

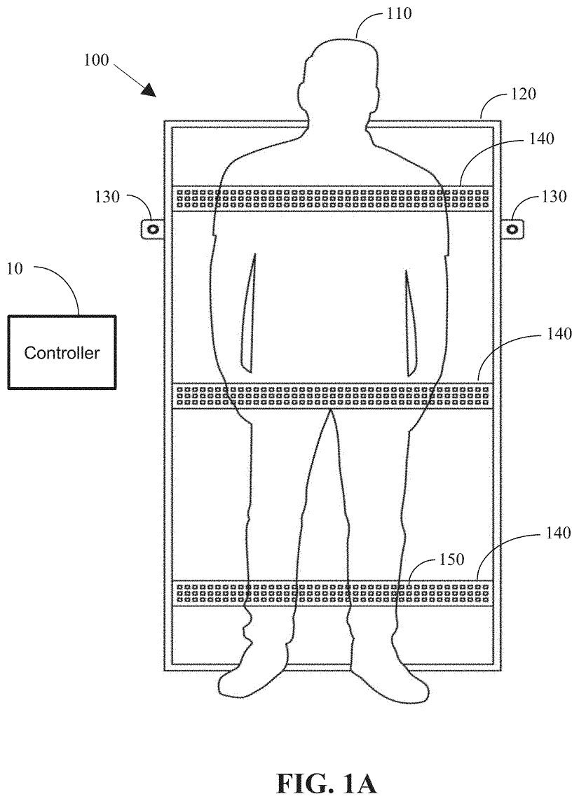

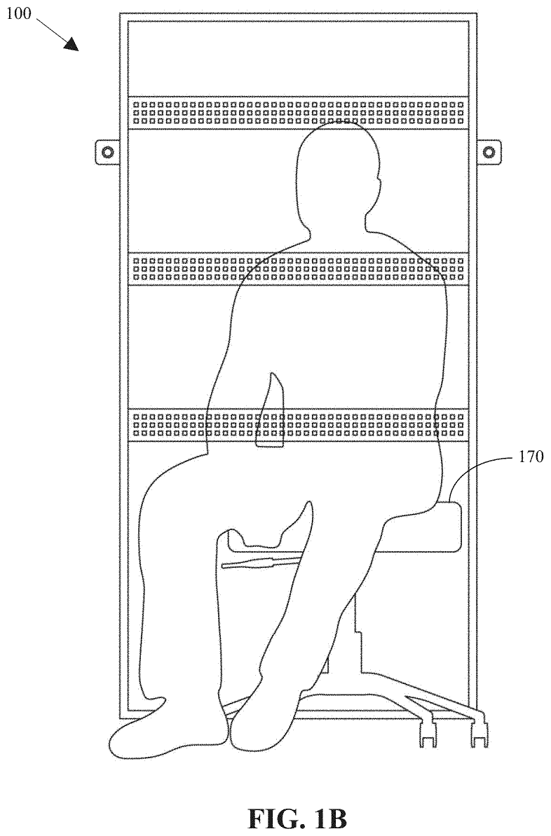

Reference is now made to FIG. 1A, which is a diagram showing an LED-based skin treatment apparatus 100 with bar-type LED assemblies, according to an embodiment of the present invention. Apparatus 100 includes a frame 120, one or more cameras 130, and one or more LED assemblies 140. Frame 120 provides the structural support for the other components of LED-based skin treatment apparatus 100.

LED-based skin treatment apparatus 100 may be positioned in front of a user 110 via a variety of methods that all fall within the scope of the invention. For example, apparatus 100 may be mounted to the wall. Apparatus 100 may have a cover that is slid over the apparatus when it is not in use, so as to render the apparatus nearly invisible. In another embodiment, apparatus 100 may include or be mounted to components such as legs and/or feet that keep apparatus 100 upright while sitting on the floor. As shown in FIG. 1B, apparatus 100 may be used with the user seated on chair 170. Alternatively, an apparatus that is to be operated while the user is seated may be smaller than apparatus 100 shown in FIGS. 1A and 1B—instead of approximating the height of a user (˜1.5 to 2 meters), the apparatus may only be about 1 meter×1 meter. In addition, this modified apparatus may be mounted 0.5 to 1 meter off of the floor. The chair may be designed to hold the user comfortably in place during a treatment session, e.g., by having rests that fit under a user's armpits to prevent the user's movement while being treated. This arrangement may facilitate treatments for 10-15 minutes on the user's back, back of neck, back of calves, and chest.

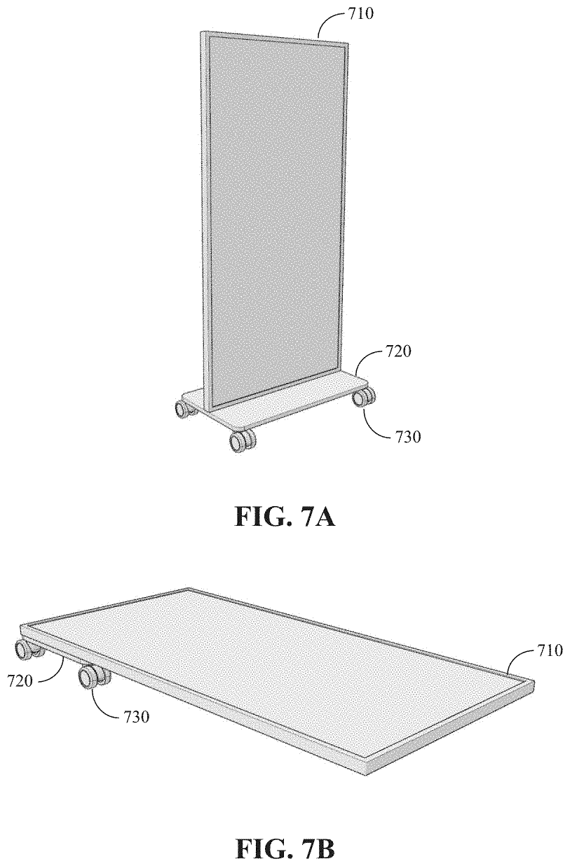

Alternatively, with reference to FIGS. 7A and 7B, apparatus 100 may be mounted to a platform 720. Platform 720 can be made movable by the inclusion of one or more wheels 730 on the bottom portion of platform 720. Furthermore, apparatus 100 may be mounted to platform 720 with a hinge that allows apparatus 100 to fold down such that it is horizontal with the floor and parallel to the top surface of the platform 720.

Camera 130 can be any type of camera, but preferably is a digital optical camera. Camera 130 provides images of user 110 to a controller 10 that may be located on or within apparatus 100, or, in fact, anywhere, communicating with the camera via a wired or wireless connection. As shown in FIG. 1A, camera 130 can be attached to an outside portion of frame 120. Alternatively, camera 130 can be located on any other portion of frame 120, such as the top or bottom portions. Alternatively, rather than being attached to frame 120 in a fixed position, camera 130 can be configured to be movable and/or tiltable with respect to frame 120. As a further alternative, camera 130 can be attached to LED assembly 140. When attached to such assembly, camera 130 can be attached in a fixed position or can be configured to be movable with respect to such assembly.

As shown in FIGS. 1A and 1B, according to an embodiment of the present invention LED assembly 140 is a rectangular, bar-type assembly. The assembly extends horizontally across the length of frame 120. The assembly is configured to be movable in the vertical direction with respect to frame 120, i.e., from the bottom of frame 120 to the top of the frame or from the top of frame 120 to the bottom of the frame. In an alternative embodiment, assembly 140 could also move in the z-direction, i.e., towards the user and/or away from the user. Assembly 140 may also tilt or rotate in the horizontal or vertical directions. If there are multiple assemblies 140, all of them can be arranged in the same vertical plane. Alternatively, each one of a plurality of assemblies 140 can be in a different vertical plane, such that each assembly can travel the full vertical range of frame 120. According to an embodiment of the present invention, apparatus 100 includes three bar-type LED assemblies 140, but can also include fewer or more and still be within the scope of the invention.

Each bar-type LED assembly 140 includes an LED array comprising a plurality of LEDs 150. The LED arrays shown in FIGS. 1A and 1B have three rows of about 40 LEDs, but each LED array may have more or fewer rows (even just one row) and more or fewer LEDs in each row. The quantity of LEDs per row and number of rows are configurable and may be based on LED size and cost, frame size, desired resolution of treatment boundaries (between lesioned and healthy skin), manufacturability, and overall cost. More specifically, having a higher resolution or density of LEDs allows better treatment accuracy, especially on the borders of the lesion. Such resolution may be achieved in a number of ways: (a) have a high density of LEDs that are individually addressable; (b) have a high density of LEDs that may be operated at partial power; (c) have precise, high-intensity, radiation source and position it directly over a portion of a lesion for a short time period. In addition, the assemblies do not have to be identical, but can have different quantities of LEDs per row and different numbers of rows.

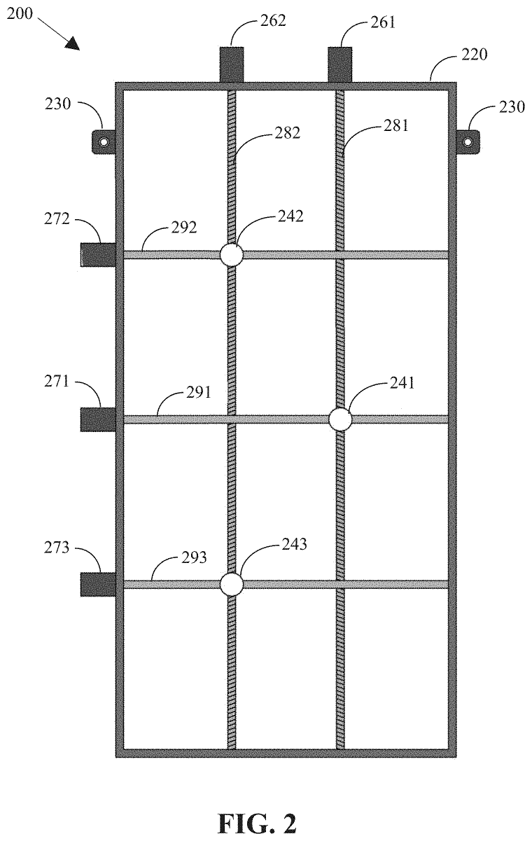

Reference is now made to FIG. 2, which is a diagram showing an LED-based skin treatment apparatus 200 with disc-type LED assemblies, according to an embodiment of the present invention. Apparatus 200 includes a frame 220, one or more cameras 230, disc-type LED assemblies 241, 242, 243, motors 261, 262, 271, 272, 273, and control rods 281, 282, 291, 292, 293. Frame 220 and camera 230 are identical to frame 120 and camera 130 that were described with respect to FIG. 1A. Similarly, the alternative embodiments of frame 120 and camera 130, as described with respect to FIG. 1A, are also alternative embodiments of frame 220 and camera 230.

As shown in FIG. 2, according to an embodiment of the present invention, LED assemblies 241, 242, 243 are disc-type assemblies. Assemblies 241, 242, 243 are configured to be movable in the vertical and horizontal directions with respect to frame 220. The movement of disc-type LED assemblies 241, 242, 243 is controlled by motors 261, 262, 271, 272, 273, which are connected to assemblies 241, 242, 243 via control rods 281, 282, 291, 292, 293. As shown in FIG. 2, LED assembly 241 is moved in the horizontal direction by motor 271 through control rod 291 and moved in the vertical direction by motor 261 through control rod 281. In an alternative embodiment, disc-type LED assemblies 241, 242, 243 could also move in the z-direction, i.e., towards the user and/or away from the user, or can tilt or rotate.

Alternatively, more than one disc-type LED assembly can be connected to one of the control rods and motors. For example, as shown in FIG. 2, disc-type LED assemblies 242 and 243 are connected to the same vertical control rod and motor. Accordingly, disc-type LED assemblies 242 and 243 are moved in the vertical direction by motor 262 through control rod 282. When multiple disc-type LED assemblies share a control rod/motor in one direction, e.g., vertical or horizontal, such assemblies also move in synchronization in the other direction, e.g., horizontal or vertical. For example, as shown in FIG. 2, disc-type LED assemblies 242 and 243 move in synchronization in the horizontal direction. Disc-type LED assemblies 242 and 243 are each connected to separate horizontal control rods, 292 and 293, respectively, and separate motors 272 and 273, respectively. Alternatively, the horizontal movement of disc-type LED assemblies 242 and 243 could be controlled by a single control rod and motor. For example, apparatus 200 could include horizontal control rod 292 and motor 272 and not include horizontal control rod 293 and motor 273, or vice versa.

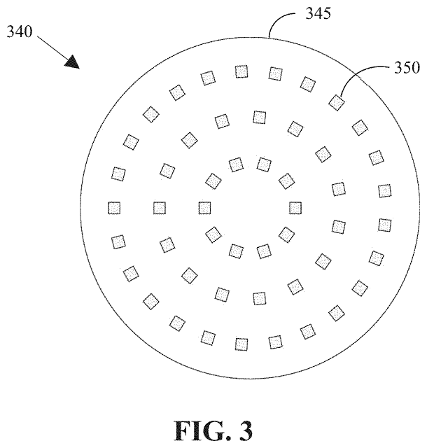

Reference is now made to FIG. 3, which is a diagram showing a disc-type LED assembly 340, according to an embodiment of the present invention. The disc-type LED assembly 340 includes a frame or housing 345 and an LED assembly composed of a plurality of LEDs 350. The LED assembly shown in FIG. 3 has fifty LEDs 350 arranged in three concentric circles with ten LEDs in the inner circle, fifteen LEDs in the middle circle, and twenty-five LEDs in the outer circle. In other embodiments, each LED assembly may have more or fewer than fifty LEDs and may have more or fewer concentric circles (or even just one circle). The quantity of LEDs per circle and number of circles are configurable and may be based on LED size and cost, disc size, desired resolution of treatment boundaries, manufacturability, and overall cost. Such resolution may be achieved as described above with respect to the bar-type assemblies. In addition, assemblies 241, 242, 243 do not have to be identical, but can have different quantities of LEDs per circle and numbers of circles. In further embodiments, LEDs 350 can be arranged in patterns other than circles, such as a grid of rows and columns (i.e., rectangular), triangular, other polygonal shapes, etc.

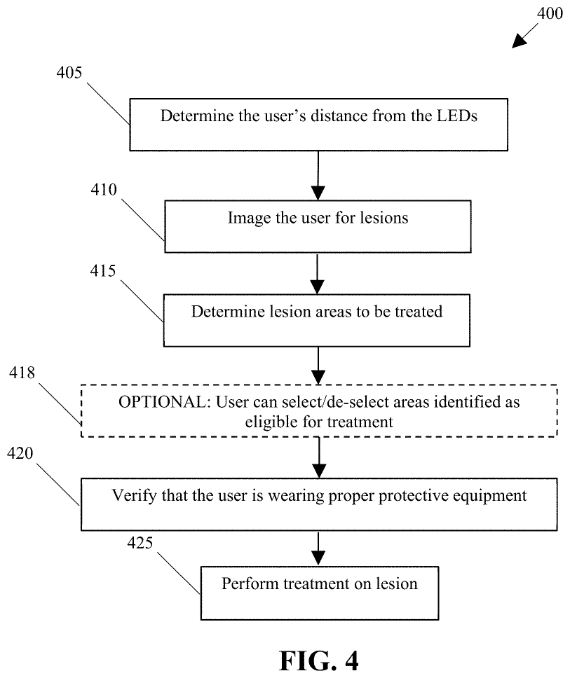

Reference is now made to FIG. 4, which is a flowchart 400 showing a process for preparing to treat skin lesions, according to an embodiment of the present invention. After the skin treatment apparatus is powered on and the user requests that the apparatus commence with a skin lesion treatment, the apparatus determines the user's distance from the LEDs in operation 405. Operation 405 can be performed by the apparatus's controller using cameras 130 or 230. In other embodiments, the apparatus may include hardware for measuring distance, such as a laser distancing device (e.g., angled laser pointers) or ultrasonic technology, which outputs the distance between the LEDs and the user to the controller. In another embodiment, cameras 130, 230 may be angled at 900 to the apparatus.

If the controller determines that the user is not at the optimal distance from the LEDs for the skin treatment, for example twelve inches, the controller instructs the user to move closer to or further from the apparatus to achieve the optimal distance for a treatment. The instruction from the controller can be communicated to the user via a visual, audible, and/or tactile notification. For example, the apparatus can display a message on a display screen that is attached to or remote from the apparatus. Alternatively, the notifications can be communicated to the user through their cell phone. The apparatus can perform the user-distance determination continuously throughout the duration of the skin treatment.

Once the user is at the optimal distance for the skin treatment, the apparatus images the user for lesion areas that are eligible for treatment, as indicated in operation 410. In an embodiment of the present invention, the imaging for lesion areas is performed by one or more of the cameras. The cameras provide optical images of the user's body, complete with skin lesions, to the controller for analysis.

Once the apparatus has imaged the lesion areas, the controller determines the lesion areas of the user's body that are to be treated, as indicated in operation 415. The controller determines areas that are to be treated by performing pattern recognition on the images obtained from the cameras. Other features may be used to determine treatment areas, including lesion color, lesion roughness, and elevation of the lesion compared to surrounding skin. In an alternative embodiment, the areas that have been determined to be treated can be communicated to the user via a user interface, located on the apparatus or remotely, such as via the user's cell phone, so that the user can select or de-select certain areas identified by the controller as eligible for skin lesion treatment.

Once the skin lesion areas have been determined, the controller determines if the user is wearing proper protective equipment in operation 420. Proper protective equipment can include, for example, protective underwear, protective eyewear, and/or other protective articles of clothing. The controller can determine whether the user is wearing the proper protective equipment based on the images obtained during the imaging operation, operation 410. Alternatively, the apparatus can use the cameras to re-image the user to determine if the user is wearing the proper protective equipment. The controller can determine if the user is wearing the proper protective equipment based on visual indications, such as color contrast between the clothes and the user's skin. Alternatively, the controller can analyze the images for particular bar and/or QR codes on the clothing, that can be used by the controller to indicate a particular article of clothing and that the clothing is authorized. The apparatus can also determine if the user has skin lesions in sensitive areas that may not warrant treatment for safety reasons. Such areas include the eyes, mouth, genitals, etc., and open wounds. Once the controller has determined that the user is wearing the proper protective equipment, the apparatus can proceed to performing the skin treatment on the lesions, as indicated in operation 425.

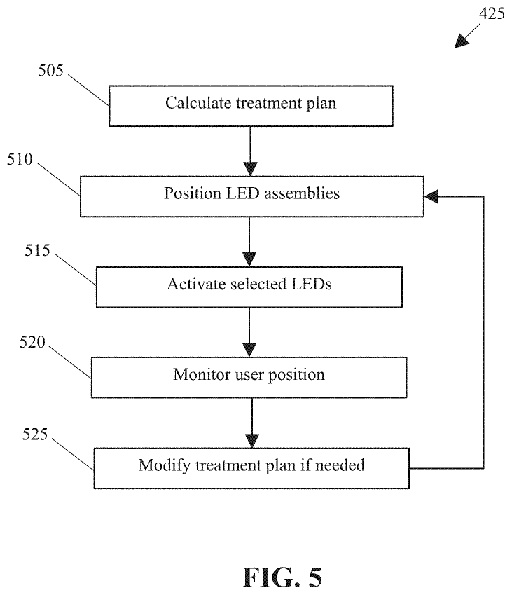

Reference is now made to FIG. 5, which is a flowchart showing a process for treating skin lesions referred to in operation 425, according to an embodiment of the present invention. The first operation in performing the skin treatment is for the controller to calculate the treatment plan in operation 505. The controller determines a treatment plan to include the dose of LED radiation to be administered and the duration of the skin treatment based on the body surface area of the areas to receive the skin treatment. The treatment plan may also take into account how long a disc stays in a certain location and the spread of energy to healthy tissue. That is, the boundaries between lesioned and healthy skin are important and can be refined by varying the proximity of the LEDs to the boundary, their energy intensity, and their stationary duration. The treatment plan calculation also determines which LED assemblies to deploy to treat each treatment area and how the LED assemblies move to cover all the treatment areas. For example, using the embodiment of FIG. 1A, the skin treatment apparatus could treat a first treatment area on the lower body with the lower LED assembly, a second treatment area on the upper body with the upper LED assembly, and a third treatment area with the middle LED assembly.

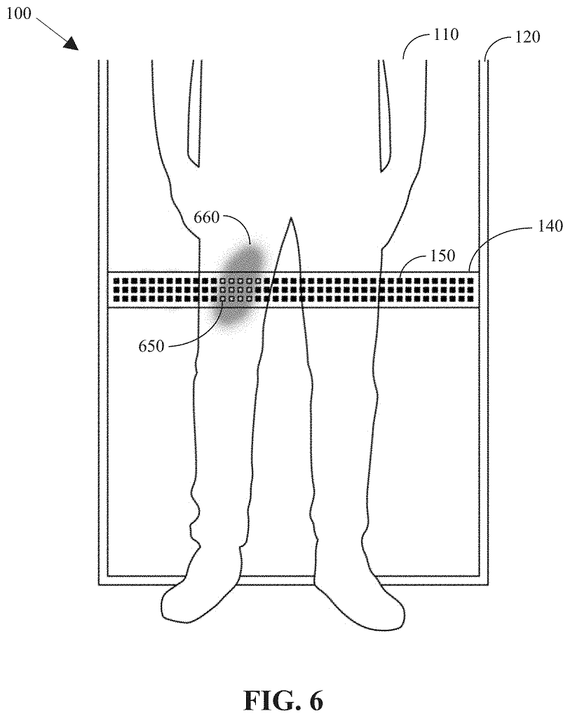

The treatment plan calculation also determines which LEDs to activate or turn on during the skin treatment so as to minimize the radiation delivered to areas of healthy skin. The controller determines which LEDs should turn on based on the geometry and quantity of the LEDs on the LED assembly being used to treat the particular treatment area. The controller's determination of which LEDs to turn on is also based on the particular geometry and size of the treatment area. For example, FIG. 6 shows LED-based skin treatment apparatus 100 with bar-type LED assemblies during a skin treatment, according to an embodiment of the present invention. In that diagram, only LEDs 650 that are over treatment area 660 will be energized or activated.

Once the controller has calculated the treatment plan, the controller instructs the appropriate motors to position the LED assemblies (whether bar or disc) according to the treatment plan, operation 510. After the LED assemblies are positioned in accordance with the treatment plan, the controller energizes or activates the LEDs according to the treatment plan in operation 515.

While the skin treatment is occurring, the apparatus monitors the user's position as shown in operation 520 and modifies the treatment plan if necessary, as shown in operation 525. The monitoring is continuous during the treatment session. For example, if the user shifts position to the left or right during the treatment, the apparatus can readjust the treatment plan to energize or activate the LEDs needed to treat the lesion areas based on the user's new position with respect to the apparatus.

The controller can also use the monitoring of the user's position to determine if the treatment should be stopped due to the user moving beyond set limits. If the user moves too much or leaves the area in front of the apparatus, the controller suspends the treatment. The controller can notify the user that the treatment has been suspended via a message on a display screen that is attached to or remote from the apparatus. Alternatively, the notification can be communicated to the user through their cell phone.

In another embodiment, the apparatus may treat a lesion in one area and image another area of the body to prepare to treat lesions in that area. In another embodiment, more than one lesion may be treated at one time. Both of these embodiments save time for the user.

In sum, an apparatus and method are presented for treating skin lesions on a user that may cover a large amount of body surface area and/or that are hard-to-reach or hard-to-see. The method obtains images of the user from a camera, determines areas of the user's body that contains skin lesions to be treated, positions an LED assembly that includes an LED array, and activates one or more LEDs in the LED array to treat the skin lesions. The LED assembly may be bar-type or disc-type, and several LED assemblies may be used at one time. The apparatus may be wall mounted, mounted on a platform, or usable while seated. The seat may restrict the user's movement for more efficient treatment.

An apparatus for treating a skin lesion on a user may include some or all of the following aspects:
A frame.
A camera capable of imaging the user.
An LED assembly coupled to the frame and including an LED array.
A controller coupled to the camera, LED assembly, and LED array. The controller obtains an image of the user from the camera, determines an area of the user's body for treating the skin lesion, positions the LED assembly, and activates one or more LEDs in the LED array to treat the skin lesion.
The apparatus may be wall-mounted and may have a sliding cover to render it nearly invisible when not in use.
The apparatus may be mounted on a platform or be used with a chair, and the chair may restrict the user's movements.
The apparatus may simultaneously treat and image different sections of the body to save time. The apparatus may simultaneously treat more than one lesion at a time.
The apparatus may have various safety mechanisms to turn off the LEDs if the user moves too much or leaves the treatment zone. Other safety mechanisms may include avoiding treating certain areas of the body and checking to make sure the user is properly attired prior to treatment.
The LEDs may be arranged to provide high resolution. The LEDs may be individually addressable, they may be operated at partial power, or they may be precise, high-intensity, radiation sources positioned directly over a portion of a lesion for a short period of time.

Aspects of the present invention may be embodied in the form of a system, a computer program product, or a method. Similarly, aspects of the present invention may be embodied as hardware, software, or a combination of both. Aspects of the present invention may be embodied as a computer program product saved on one or more computer-readable media in the form of computer-readable program code embodied thereon.

The computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language. The program code may execute on a single computer, or on a plurality of computers. The computer may include a processing unit in communication with a computer-usable medium, where the computer-usable medium contains a set of instructions, and where the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. An apparatus for treating a skin lesion on a user, comprising:
a frame;
a camera capable of imaging the user;
a light emitting diode (LED) assembly coupled to the frame and comprising an LED array; and
a controller operatively coupled to the camera, LED assembly, and LED array;
wherein the controller is configured to:
obtain an image of the user from the camera;
determine an area of the user's body for treating the skin lesion;
position the LED assembly; and
activate one or more LEDs in the LED array to treat the skin lesion.

2. The apparatus of claim 1, wherein the apparatus further comprises hardware for measuring distance.

3. The apparatus of claim 1, wherein the LED assembly coupled to the frame is a bar-type assembly.

4. The apparatus of claim 1, wherein the LED assembly coupled to the frame is a disc-type assembly.

5. The apparatus of claim 4, further comprising:
a control rod operatively coupled to the LED assembly; and
a motor operatively coupled to the control rod.

6. The apparatus of claim 1, wherein the camera is mounted to the frame.

7. The apparatus of claim 1, wherein the camera is mounted to the LED assembly.

8. The apparatus of claim 1, further comprising:
a platform with an upper surface and a bottom surface; and
one or more wheels mounted to the bottom surface of the platform,
wherein the frame is mounted to the upper surface of the platform.

9. The apparatus of claim 1, wherein the controller is further configured to verify that the user is wearing protective equipment.

10. The apparatus of claim 1, wherein the controller is further configured to avoid treating sensitive areas of the body.

11. The apparatus of claim 1, wherein the controller is further configured to determine which LEDs to activate based on the determination of the area of the user's body to treat the skin lesion.

12. The apparatus of claim 10, wherein the determination of the area of the user's body to treat the skin lesion identifies the specific LEDs needed to treat the skin lesion.

13. The apparatus of claim 11, wherein the controller is further configured to activate only the specific LEDs needed to treat the skin lesion.

14. The apparatus of claim 1, wherein the LED array comprises a high density of LEDs that are individually addressable.

15. The apparatus of claim 1, wherein the LED array comprises a high-intensity radiation source that is positioned substantially directly over a portion of a lesion for a predetermined time period.

16. The apparatus of claim 1, wherein the LED array comprises a high density of LEDs that are operated at partial power.

17. The apparatus of claim 1, wherein the user is treated while sitting in a chair.

18. The apparatus of claim 17, wherein the chair restricts movement of the user.

19. The apparatus of claim 1, wherein the frame is configured to be mounted to a wall.

20. The apparatus of claim 19, further comprising a cover configured to slide over the frame.

21. The apparatus of claim 1, wherein the controller is further configured to simultaneously treat a lesion in one area of the user's body and image another area of the user's body for future treatment.

22. The apparatus of claim 1, wherein the controller is further configured to simultaneously treat more than lesion on the user's body.

23. A method for treating a skin lesion on a user, comprising:
obtaining an image of the user from a camera;
determining an area of the user's body for treating the skin lesion;
positioning an LED assembly comprising an LED array; and
activating one or more LEDs in the LED array to treat the skin lesion.

24. The method of claim 23, further comprising determining which LEDs to activate based on the determination of the area of the user's body for treating the skin lesion.

25. The method of claim 24, wherein determining which LEDs to activate based on the determination of the area of the user's body for treating the skin lesion identifies the specific LEDs needed to treat the skin lesion.

26. The method of claim 25, wherein activating one or more LEDs in the LED array comprises activating only the specific LEDs needed to treat the skin lesion.

27. The method of claim 23, further comprising verifying that the user is wearing protective equipment.

28. The method of claim 23, further comprising determining a distance between the user and the LEDs.

29. The method of claim 28, further comprising turning off the LEDs if the distance between the user and the LEDs is less than a pre-determined threshold.

30. The method of claim 28, further comprising turning off the LEDs if the distance between the user and the LEDs is more than a pre-determined threshold.

31. The method of claim 23, further comprising determining whether the user is moving.

32. The method of claim 31, further comprising turning off the LEDs if the user moves more than a pre-determined amount.

33. The method of claim 31, further comprising: determining if treatment should be stopped due to the user moving beyond set limits; and notifying the user.

34. The apparatus of claim 1, wherein the LED assembly coupled to the frame comprises a plurality of LEDs arranged in a geometric pattern.

35. The apparatus of claim 1, wherein the apparatus is tiltable to accommodate user position.
